(19) United States
(12) Patent Application Publication
(10) Pub. No.: US 2004/0137024 A1
(43) Pub. Date: Jul. 15, 2004

(54) Smoothing out wrinkles with a fragrancing composition

(75) Inventors: Anne Abriat, Paris (FR); Brigitte Petit, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/694,869

(22) Filed: Oct. 29, 2003

Related U.S. Application Data
(60) Provisional application No. 60/426,374, filed on Nov. 15, 2002.

(30) Foreign Application Priority Data
Oct. 29, 2002 (FR) 0213523

(51) Int. Cl. A61K 7/00

(52) U.S. Cl. 424/401

(57) ABSTRACT

Smoothing out expression wrinkles and/or lines of the face by topical application to facial skin of a composition containing, in a physiologically acceptable medium, a fragrancing composition containing from 5% to 10% by weight of essential oils and from 90% to 95% by weight of a mixture comprising:

(a) 10% to 15% by weight of alcohols,

(b) 10% to 15% by weight of aldehydes,

(c) 25% to 30% by weight of esters,

(d) 20% to 30% by weight of at least one compound selected from the group consisting of: musks and ketones, and

(e) 5% to 10% by weight of solvents.

### REFERENCE TO PRIOR APPLICATIONS

This application claims priority to U.S. provisional application No. 60/426,374 filed Nov. 15, 2002, and to French patent application 0213523 filed Oct. 29, 2002, both incorporated herein by reference.

### FIELD OF THE INVENTION

The present invention relates to a process for preventing and/or smoothing out wrinkles such as expression wrinkles and/or for relaxing the lines of the face, comprising the topical application to facial skin of a composition comprising, in a physiologically acceptable medium, a fragrancing composition, this fragrancing composition resulting in, when it is introduced in a proportion of 0.5% by weight into 2 ml of a topical preparation applied to the part of the skin located between the nose and the top lip of a human individual subjected to stress, a reduction in the muscular activity of the trapezium muscle, measured by electromyography, compared with the unfragranced topical preparation tested under the same conditions.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BRIEF DESCRIPTION OF THE DRAWINGS

FIG. 1: FIG. 1 describes the placement of sensors coated with conductive gel on individuals for measuring the activity of the trapezius muscle by electromyography.

FIG. 2: FIG. 2 shows that individuals to whom a fragranced cream was applied showed significantly lower muscular tension values than individuals to whom an unfragranced cream base was applied.

### BACKGROUND OF THE INVENTION

Women, and even men, currently have a tendency to wish to look youthful for as long as possible and consequently seek to fade out the signs of ageing on the skin, which are reflected in particular by wrinkles and fine lines. In this respect, the media and the fashion world report about products intended to keep the skin radiant and wrinkle-free for as long as possible, which are signs of youthful skin, and all the more so since the physical appearance acts on the psyche and/or on the morale.

Hitherto, wrinkles and fine lines were treated using cosmetic products containing active agents acting on the skin, for example by moisturizing it or by improving its cell renewal or alternatively by promoting the synthesis of collagen, of which skin tissue is composed.

Although these treatments make it possible to act on the wrinkles and fine lines caused by chronological or intrinsic ageing, and also on those caused by photoageing, they have no effect on expression wrinkles and fine lines, which require an intervention on the contractile muscle component of the wrinkles present in the skin.

Hitherto, the only means commonly used for acting on expression wrinkles is botulinum toxin, which is especially injected into the wrinkles of the glabella, which are the wrinkles between the eyebrows (see J. D. Carruters et al., J. Dermatol. Surg. Oncol., 1992, 18, pp. 17-21).

Various compounds capable of affording a muscle-relaxing effect when they are applied topically to the skin have been proposed, thus making it possible to act on expression wrinkles via another route. Among these compounds, mention may be made especially of antagonists of the receptors associated with the calcium channels (FR-2 793 681), and in particular manganese and its salts (FR-2 809 005) and alverine (FR-2 798 590); and agonists of the receptors associated with the chlorine channels, including glycine (EP-0 704 210) and certain extracts of Iris pallida (FR-2 746 641).

However, these natural compounds and extracts are not always easy to formulate in anti-ageing compositions.

### DETAILED DESCRIPTION OF THE PREFERRED EMBODIMENTS

The inventors have now discovered, surprisingly, that certain fragrancing compositions have relaxing properties on certain striated muscles, making it possible to use them for relaxing the muscles of the face and thus their introduction into compositions, particularly cosmetic compositions, for combating expression wrinkles and the like. As used herein, cosmetic compositions are those that may be topically applied to facial skin.

To the inventor's knowledge, it has never as yet been proposed to use fragrances for this purpose, despite the development of aromatherapy.

One subject of the invention is thus a process for preventing and/or smoothing out expression wrinkles and/or for relaxing the lines of the face, comprising the topical application to facial skin of a cosmetic composition comprising, in a physiologically acceptable medium, a fragrancing composition comprising from 5% to 10% by weight of essential oils and from 90% to 95% by weight of a mixture comprising:

(a) 10% to 15% by weight of alcohols,

(b) 10% to 15% by weight of aldehydes,

(c) 25% to 30% by weight of esters,

(d) 20% to 30% by weight of at least one compound selected from the group consisting of: musks and ketones, and

(e) 5% to 10% by weight of solvent(s),

this fragrancing composition resulting in, when it is introduced in a proportion of 0.5% by weight into 2 ml of a topical preparation applied to the part of the skin located between the nose and the top lip of a human individual subjected to stress, a reduction in the muscular activity of the trapezius muscle, measured by electromyography, compared with the unfragranced topical preparation tested under the same conditions.

A subject of the present invention is also the use, as an active agent for preventing and/or smoothing out expression wrinkles and/or for relaxing lines, of a fragrancing composition comprising from 5% to 10% by weight of essential oils and from 90% to 95% by weight of a mixture comprising:

(a) 10% to 15% by weight of alcohols,

(b) 10% to 15% by weight of aldehydes,

(c) 25% to 30% by weight of esters,

(d) 20% to 30% by weight of at least one compound selected from the group consisting of: musks and ketones, and

(e) 5% to 10% by weight of solvents,

this fragrancing composition resulting in, when it is introduced in a proportion of 0.5% by weight into 2 ml of a topical preparation applied to the part of the skin located between the nose and the top lip of a human individual subjected to stress, a reduction in the muscular activity of the trapezius muscle, measured by electromyography, compared with the unfragranced topical preparation tested under the same conditions.

The electromyography test that can be used to detect a reduction in the muscular activity of the trapezius muscle is described in greater detail in Example 1. To perform this test, the fragrancing composition is formulated in a topical preparation, which may be of any chemical composition provided that this preparation is also used as a control in this test. The same reference topical preparation may be used to test different fragrancing compositions. In contrast with preferred compositions according to the invention, the topical preparation does not necessarily satisfy all the requirements of a commercial cosmetic composition, in particular as regards its texture and its stability over time.

The composition used according to the invention is most preferably suitable for topical application to the skin and thus contains a physiologically acceptable medium, i.e. a medium that is compatible with the skin and optionally with its integuments (eyelashes, nails and hair) and/or mucous membranes. It may be, for example, a skincare, cleansing or makeup composition.

It comprises an amount of fragrancing composition that is sufficient to obtain the desired effect and preferably from 0.1% to 25% by weight relative to the total weight of the cosmetic composition.

This composition is advantageously applied to individuals with expression wrinkles and fine lines. These wrinkles and fine lines are mainly located radially around the mouth and/or the eyes and/or horizontally on the forehead and/or in the space between the eyebrows. In addition, since its beneficial effect on the skin is believed to act via inhalation of the fragrancing composition it contains, the composition according to the invention is also preferably applied to the part of the individuals skin located between the nose and the mouth, or only to the part of the individual's skin located between the nose and the mouth.

The term “fragrancing composition” used herein denotes a mixture of odoriferous materials whose vapour pressure is less than the atmospheric pressure at 25° C. and which are generally liquid at 25° C.

The constituents of the fragrancing composition used according to the invention include those described especially in S. Arctander, Perfume and Flavor Chemicals (Montclair, N.J., 1969), in S. Arctander, Perfume and Flavor Materials of Natural Origin (Elizabeth, N.J., 1960) and in “Flavor and Fragrance Materials—1991”, Allured Publishing Co. Wheaton, III. USA.

They may be natural products (e.g., essential oils, absolutes, resinoids, resins or concretes) and/or synthetic products (e.g., hydrocarbons, alcohols, aldehydes, ketones, ethers, acids, esters, acetals, ketals, nitriles, which may be saturated or unsaturated, and aliphatic or cyclic).

Examples of essential oils comprise the essential oils of lemon, of orange, of aniseed, of bergamot, of rose, of geranium, of ginger, of neroli, of basil, of rosemary, of cardamom, of camphor, of cedar, of camomile, of sandalwood, and of sage, and mixtures thereof, this list not being limiting.

Examples of other compounds of which the fragrancing composition according to the invention may be composed are, especially: geraniol, geranyl acetate, farnesol, borneol, bornyl acetate, linalool, linalyl acetate, linalyl propionate, linalyl butyrate, tetrahydrolinalool, citronellol, citronellyl acetate, citronellyl formate, citronellyl propionate, dihydromyrcenol, dihydromyrcenyl acetate, tetrahydromyrcenol, terpineol, terpinyl acetate, nopol, nopyl acetate, nerol, neryl acetate, 2-phenylethanol, 2-phenylethyl acetate, benzyl alcohol, benzyl acetate, benzyl salicylate, styrallyl acetate, benzyl benzoate, amyl salicylate, dimethylbenzylcarbinol, trichloromethyl-phenylcarbinyl acetate, p-tert-butylcyclohexyl acetate, isononyl acetate, vetiveryl acetate, vetiverol, α-hexylcinnamaldehyde, 2-methyl-3-(p-tert-butylphenyl)-propanal, 2-methyl-3-(p-isopropylphenyl)propanal, 3-(p-tert-butylphenyl)propanal, 2,4-dimethylcyclohex-3-enylcarboxaldehyde, tricyclodecenyl acetate, tricyclodecenyl propionate, 4-(4-hydroxy-4-methylpentyl)-3-cyclohexenecarboxaldehyde, 4-(4-methyl-3-pentenyl)-3-cyclohexenecarboxaldehyde, 4-acetoxy-3-pentyltetrahydropyran, 3-carboxymethyl-2-pentylcyclopentane, 2-n-heptylcyclopentanone, 3-methyl-2-pentyl-2-cyclo-pentenone, menthone, carvone, tagetone, geranyl acetone, n-decanal, n-dodecanal, 9-decen-1-ol, phenoxyethyl isobutyrate, phenylacetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, aubepinonitrile, aubepine, heliotropin, coumarin, eugenol, vanillin, diphenyl ether, citral, citronellal, hydroxycitronellal, damascone, ionones, methylionones, isomethylionones, solanone, irones, cis-3-hexenol and its esters, indane musks, tetralin musks, isochroman musks, macrocyclic ketones, macrolactone musks and ethylene brassylate, and mixtures thereof.

The fragrancing composition according to the invention also generally comprises one or more solvents that are standard in fragrances, which may be chosen especially from: ethanol, isopropanol, diethylene glycol monoethyl ether, dipropylene glycol, diethyl phthalate, triethyl citrate, isopropyl myristate, etc.

The cosmetic composition containing the fragrancing composition according to the invention may be in any presentation form conventionally used for topical application and especially in the form of aqueous gels and aqueous or aqueous-alcoholic solutions. By adding a fatty or oily phase, it may also be in the form of dispersions of the lotion or serum type, emulsions of liquid or semi-liquid consistency of the milk type, obtained by dispersing a fatty phase in an aqueous phase (O/W) or conversely (W/O), or suspensions or emulsions of soft, semi-solid or solid consistency of the cream or gel type, or multiple emulsions (W/O/W or O/W/O emulsions), microemulsions, vesicular dispersions of ionic and/or nonionic type, or wax/aqueous phase dispersions. These compositions are prepared according to the usual methods.

According to one preferred embodiment of the invention, the cosmetic composition is in the form of an emulsion.

In this case, the proportion of the oily phase of the emulsion may range, for example, from 5% to 80% by weight and preferably from 5% to 50% by weight relative to the total weight of the composition. The fatty substances, emulsifiers and co-emulsifiers used in the composition in emulsion form are selected from the group consisting of those conventionally used in cosmetics or dermatology. The emulsifier and the co-emulsifier are generally present in the composition in a proportion ranging from 0.3% to 30% by weight and preferably from 0.5% to 20% by weight relative to the total weight of the composition. The emulsion may also contain lipid vesicles.

Fatty substances that may be used include oils and especially mineral oils (liquid petroleum jelly), oils of plant origin (avocado oil, rice bran oil, wheatgerm oil, apricot kernel oil, soybean oil, coconut oil, palm oil and rapeseed oil), oils of animal origin (lanolin), synthetic oils (perhydrosqualene and hydrogenated polyisobutene), silicone oils (cyclomethicone) and fluoro oils (perfluoropolyethers). Fatty substances that may also be used include fatty alcohols such as cetyl alcohol, stearyl alcohol and octyldodecanol, fatty acids, fatty acid esters such as pentaerythrityl tetraethylhexanoate, waxes and gums, and in particular silicone gums.

Examples of emulsifiers and co-emulsifiers that may be mentioned include fatty acid esters of polyethylene glycol, such as PEG-100 stearate, PEG-40 stearate and PEG-20 stearate; fatty acid esters of polyols, such as glyceryl stearate, sorbitan tristearate and the oxyethylenated sorbitan stearates sold under the trade names Tween® 20 or Tween® 60, for example; and mixtures thereof.

In a known manner, the composition used according to the invention may also contain adjuvants, such as hydrophilic or lipophilic gelling agents, hydrophilic or lipophilic active agents, preserving agents, fragrances, fillers, screening agents, pigments, odour absorbers and dyestuffs. The amounts of these various adjuvants may be those conventionally used in the field under consideration, for example from 0.01% to 20% of the total weight of the composition. Depending on their nature, these adjuvants may be introduced into the fatty phase, into the aqueous phase or into the lipid vesicles. In any case, these adjuvants and the proportions thereof will be chosen so as not to harm the desired properties of the fragrancing composition according to the invention.

Hydrophilic gelling agents that may be mentioned in particular include carboxyvinyl polymers (carbomer), acrylic copolymers such as acrylate/alkylacrylate copolymers, polyacrylamides and in particular acrylamide/sodium acryloyldimethyltaurate copolymer and the ammonium salt of polyacryloyldimethyl taurate, polysaccharides, natural gums and clays, and lipophilic gelling agents that may be mentioned include modified clays, for instance bentones, metal salts of fatty acids and hydrophobic silica.

Fillers that may be mentioned especially include talc and silica, and also polyamide fibres.

As active agents, the cosmetic composition according to the invention also advantageously contains at least one compound that may be chosen especially from: desquamating agents; moisturizers; depigmenting or propigmenting agents; anti-glycation agents; NO-synthase inhibitors; agents for stimulating the synthesis of dermal or epidermal macromolecules and/or for preventing their degradation; agents for stimulating fibroblast and/or keratinocyte proliferation or for stimulating keratinocyte differentiation; muscle relaxants; tensioning agents; antipollution agents and/or free-radical scavengers; agents acting on the capillary circulation; agents acting on the energy metabolism of cells; and mixtures thereof.

Active agents that are preferred for use according to the present invention comprise one or more compounds selected from the group consisting of: manganese gluconate; an extract of wild yam containing diosgenin; a ceramide; a retinoid selected from the group consisting of: retinyl palmitate, retinyl caprylate and retinol; and acetyl trifluoromethylphenyl valylglycine.

The invention will now be illustrated with the non-limiting examples that follow.

### EXAMPLES

### Example 1

### Demonstration of the Relaxing Effect on Striated Muscle

Experimental Protocol

The effect on the trapezius muscle of a fragrancing composition formulated in a topical preparation in cream form was evaluated.

The fragrancing composition tested consisted of a mixture of:

natural products 7%, including essential oil of bergamot, mandarin and ylang-ylang

synthetic products 93%, including

| | |
|---|---|
| aliphatic and aromatic alcohols | 13% |
| aliphatic and aromatic aldehydes | 13.50% |
| aliphatic and aromatic esters | 28.50% |
| synthetic ketones and musks | 25% |
| solvents | 9% |
| various (monoterpenes, saturated products) | 4% |

The study was performed on 40 individuals between 21 and 50 years old. The individuals were divided into 2 groups of 20 individuals. One group tested the fragranced cream and the second group the unfragranced base. The fragrance presented was of good intensity, neither too strong nor too weak.

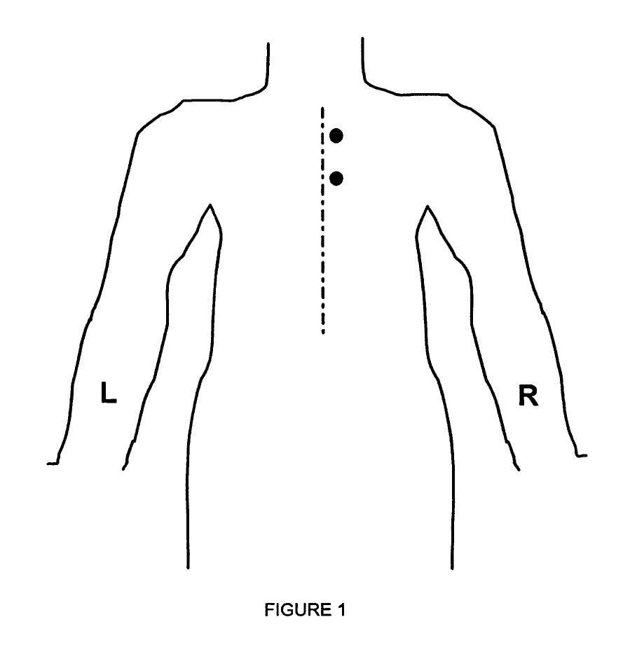

Sensors coated with conductive gel were placed on the individuals according to the scheme indicated in FIG. 1 attached hereto, in which the dotted line illustrates the vertebral column of the individual and the discs denote the positions of the sensors, the individual being viewed from the back. The target areas of skin were wiped with alcohol before application, to improve the signal-to-noise ratio.

After the sensors were placed on the individuals, the said individuals were subjected to a first series of tests lasting 30 minutes to familiarize them with the procedure. Each individual then applied 2 ml of cream by finger to the area of skin located between the top lip and the nose.

The activity of the trapezius muscle of each individual was measured by electromyography using sensors installed beforehand, with the aid of a Physiolab I-330 C-2 machine from J&J Engineering Inc. during each of the three-minute periods below:

first period (rest): the individual was placed, with the eyes open, before a pleasant scene

second period (training): the individual had to count coloured squares following each other every 10 seconds

third period (stress): the individual had to name the colours of series of words passing before him at a rate of one per second, the words corresponding to colours different from those in which they were written. In the middle of the test, this was complicated by moving the word on the screen and adding stressful sounds such as breaking glass, the screeching of tyres, etc.

fourth period (recovery): as for the second period.

The data were filtered using a 100-400 Hz filter and then integrated and their mean was calculated to obtain a mean value in microvolts corresponding to each of the above four periods.

The value corresponding to the first period, which is not significantly different from one group to another, was used as covariance to eliminate the differences between the groups and between the individuals. This made it possible to obtain a finer statistical analysis and to give a better analysis of the effects. The EMG data were analysed by means of repeated covariance measurements (ANCOVA) under 3 conditions (easy tasks, stress, return to normal). The significance was established at 0.1% for the differences between the groups and between the individuals.

Results

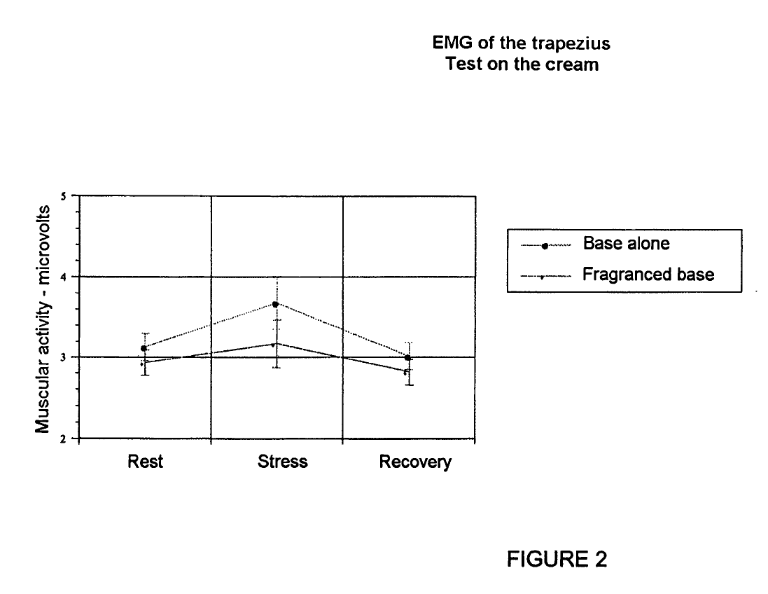

As illustrated in FIG. 2, during the stress period, the individuals to whom the fragranced cream was applied showed significantly lower muscular tension values than the individuals to whom the unfragranced cream base was applied. The difference is significant to 0.1%.

The muscle-relaxing effect of the fragrancing composition used according to the invention was thus demonstrated.

### Example 2

### Demonstration of the In Vivo Anti-Wrinkle Effect

The study was performed on two paired groups of 53 individuals each, applying for 2 weeks: for the first group (A), a cosmetic composition (A) containing the fragrance subjected to the test of Example 1, and, for the second group (B), an identical cosmetic composition (B) but containing a standard fragrance of pleasant odour.

A sealed questionnaire was given to the individuals at the end of the treatment to allow them to self-assess the performance qualities of the product. It comprised several assertions with respect to which the individuals had to indicate whether they were entirely, relatively, relatively not or not at all in agreement.

The results are collated in Table 1 below:

TABLE 1
Perception of the fragranced compositions

| | Composition A | Composition B | Significance |
|---|---|---|---|
| | Total % of “entirely in | | |
| | agreement” and “relatively in | | |
| | agreement” | | |
| Makes the skin smooth | 92 | 70 | p < 0.05 |
| Makes the skin firm | 86 | 68 | p < 0.05 |
| Gives the skin tonicity | 91 | 77 | p < 0.05 |
| | % of “entirely in agreement” | | |
| Prevents ageing | 34 | 17 | p < 0.05 |

These results demonstrate a perceived efficacy on the signs of ageing, which is greater for composition A containing a fragrance according to the invention than for composition B containing a standard fragrance.

Moreover, between 70% and 80% of the individuals of groups A and B assessed the fragrances contained in compositions A and B as being pleasant, of good intensity and suitable for anti-wrinkle care (non-significant difference between the groups).

### Example 3

### Cosmetic Compositions

The compositions below were prepared in a manner that is conventional to those skilled in the art. All the percentages given are percentages by weight. The fragrance has the composition indicated in Example 1.

A. Anti-wrinkle cream

| FATTY PHASE | |
|---|---|
| Polyethylene glycol stearate | 1% |
| Glyceryl stearate | 2% |
| Cetyl alcohol | 1% |
| Stearic acid | 2% |
| Apricot oil | 5% |
| Myristyl myristate | 5% |
| Cyclopentadimethylsiloxane | 5% |
| Fragrance | 0.5% |
| AQUEOUS PHASE | |
| Water | qs 100% |
| Preserving agents | qs |
| Triethanolamine | qs pH 6.5 |
| Glycerol | 5 |
| Plant extracts (active agents) | 5 |

B. Makeup-removing milk

| | |
|---|---|
| FATTY PHASE | |
| Isopropyl palmitate | 8% |
| Mineral oil | 17% |
| Fragrance | 0.3% |
| AQUEOUS PHASE | |
| Carbomer | 1% |
| Water | qs 100% |
| Glycerol | 5% |
| Sodium hydroxide | qs pH 6.5 |

C. Tonic lotion

| Water | qs 100% |
|---|---|
| Glycerol | 5% |
| Preserving agents | qs |
| Plant extract | 3% |
| Fragrance | 0.01% |

D. Foaming composition

| A) Water | qs 100% |
|---|---|
| Glycerol | 3% |
| Preserving agents | qs |
| B) Sodium lauryl ether sulphate | 15% |
| Alkylpolyglucoside | 6% |
| C) Acrylate/steareth/methacrylate copolymer | 5% |
| D) Water | 1.2% |
| Sodium hydroxide | 0.21% |

E. Fragrancing cream

| | |
|---|---|
| Liquid paraffin | 43% |
| Polydimethylsiloxane | 0.3% |
| Paraffin | 6% |
| Ozokerite | 7% |
| Liquid paraffin | 1% |
| Nylon powder | 20% |
| Fragrance | 20% |
| Polytrap | 3% |
| Nylon powder | 0.5% |

The above description and illustrative compositions and their uses thus make it possible to prevent and/or smooth out expression wrinkles and/or relax the lines of the face, to smooth out the surface of the skin and to reduce the depth of the expression wrinkles and fine lines. In this regard one of ordinary skill in the art is capable, in view of this disclosure, of adjusting the application amount of cosmetic composition and/or the amount/concentration of fragrancing composition such that the above objects (e.g., prevent and/or smooth out expression wrinkles and/or relax the lines of the face, to smooth out the surface of the skin and to reduce the depth of the expression wrinkles and fine lines) can be accomplished without undue experimentation. That is, the above written description of the invention provides a manner and process of making and using it such that any person skilled in this art is enabled to make and use the same, this enablement being provided in particular for the subject matter of the appended claims, which make up a part of the original description.

As used above, the phrase “selected from the group consisting of” includes mixtures of the specified materials.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for preventing and/or smoothing out expression wrinkles and/or for relaxing the lines of the face, comprising the topical application to facial skin of a cosmetic composition comprising, in a physiologically acceptable medium, a fragrancing composition comprising from 5% to 10% by weight of essential oils and from 90% to 95% by weight of a mixture comprising:
(a) 10% to 15% by weight of alcohols,
(b) 10% to 15% by weight of aldehydes,
(c) 25% to 30% by weight of esters,
(d) 20% to 30% by weight of at least one compound selected from the group consisting of musks and ketones, and
(e) 5% to 10% by weight of solvents,
the fragrancing composition resulting in, when it is introduced in a proportion of 0.5% by weight into 2 ml of a topical preparation applied to that part of the skin located between the nose and the top lip of a human individual subjected to stress, a reduction in the muscular activity of the trapezius muscle, measured by electromyography, compared with an unfragranced topical preparation tested under the same conditions.

2. The process according to claim 1, wherein the cosmetic composition is selected from the group consisting of skincare, cleansing and makeup compositions.

3. The process according to claim 1, wherein the fragrancing composition represents from 0.01% to 25% of the total weight of the cosmetic composition.

4. The process according to claim 1, wherein said cosmetic composition further comprises at least one compound selected from the group consisting of desquamating agents; moisturizers; depigmenting agents; propigmenting agents; anti-glycation agents; NO-synthase inhibitors; agents for stimulating the synthesis of dermal or epidermal macromolecules and/or for preventing their degradation; agents for stimulating fibroblast and/or keratinocyte proliferation or for stimulating keratinocyte differentiation; muscle relaxants; tensioning agents; antipollution agents and/or free-radical scavengers; agents acting on the capillary circulation; agents acting on the energy metabolism of cells; and mixtures thereof.

5. The process according to claim 4, wherein said cosmetic composition further comprises at least one compound selected from the group consisting of manganese gluconate; an extract of wild yam containing diosgenin; a ceramide; retinyl palmitate; retinyl caprylate; retinol; and acetyl trifluoromethylphenyl valylglycine.

6. The process according to claim 1, wherein said composition is applied to individuals with expression wrinkles and fine lines.

7. The process according to claim 1, wherein the composition is applied to the part of the skin located between the nose and the mouth.

8. The process according to claim 1, for smoothing out expression wrinkles.

9. The process according to claim 1, for relaxing the lines of the face.

10. The process according to claim 1, wherein the composition is applied only to the part of the skin located between the nose and the mouth.
